(19) United States
(12) Patent Application Publication
(10) Pub. No.: US 2020/0043589 A1
(43) Pub. Date: Feb. 6, 2020

(54) SMARTPHONE APP FOR DOSE CAPTURE AND METHOD

(71) Applicant: Becton, Dickinson and Company, Franklin Lakes, NJ (US)

(72) Inventor: J. RICHARD GYORY, Sudbury, MA (US)

(73) Assignee: Becton, Dickinson and Company, Franklin Lakes, NJ (US)

(21) Appl. No.: 16/498,584

(22) PCT Filed: Apr. 19, 2018
(86) PCT No.: PCT/US18/28322

§ 371 Date: Sep. 27, 2019

Related U.S. Application Data
(60) Provisional application No. 62/487,997, filed on Apr. 20, 2017.

(51) Int. Cl. G16H 20/17; G06F 16/583; G16H 30/40; G16H 70/40; G16H 30/20; A61M 5/315; A61J 1/03; G06K 9/22; G06K 9/62

(52) U.S. Cl. CPC G16H 20/17; G06F 16/5838; G06F 16/5854; G16H 30/40; G16H 70/40; A61M 2005/3126; A61M 5/315; A61J 1/03; G06K 9/22; G06K 9/6201; A61J 2205/20; G16H 30/20

(57) ABSTRACT

A handheld electronic device, such as a smartphone, and an app capture an image of a medication or delivery device at the time of delivery with a date and time indictor or stamp. The image is analyzed and the dosage calculated from the captured image. The device stores the data corresponding to the dosage and time and compares the data with stored information relating to the patient to record compliance with the prescribed medication and delivery protocol. The data is used to record total usage of the medication over a predetermined period of time and determine a time schedule for resupplying the medication and a delivery method when the total medication usage exceeds a predetermined amount.

This application claims priority to U.S. Provisional Application Ser. No. 62/487,997, filed Apr. 20, 2017, which is hereby incorporated by reference in its entirety.

### FIELD OF THE INVENTION

The present invention is directed to a portable, handheld electronic device, such as a smartphone, and an application to assist with medication identification and verification, and to track and record medication doses taken by a patient. The invention is further directed to a method using the device to identify a medication and calculate and record a sequence of dosages delivered to or taken by a patient.

### BACKGROUND OF THE INVENTION

Medication non-adherence is an issue of global importance, particularly with regard to diabetes care. 50% of all patients do not take their medication as prescribed. Non-adherence directly contributes to hundreds of thousands of deaths and billions of dollars in avoidable medical and related costs.

There are smartphone apps that use a picture of a prescription label to help a patient reorder when their dose is low. However, these apps do not directly identify the medication or the dose prior to the patient taking the medication, and are not useful for syringes or pen injectors.

There are medication reminder apps that alarm when it is time to take medication, but these are limited in usefulness, and do not directly confirm a medication type, or concentration, and do not affirmatively record medication doses delivered to the patient.

While the prior devices, apps and methods are generally suitable for the intended purpose, there is a continuation need for methods and devices that monitor medication usage.

### SUMMARY OF THE INVENTION

The invention is directed to a portable electronic device that is able to capture an image of a medication and/or medication delivery device and calculate a dosage delivered to a patient based on the captured image. The device stores the information relating to the dosages and records the information producing a record of the dosage and time for compliance with a prescribed medication delivery protocol.

Embodiments of the present invention provide an electronic device, such a smartphone having a smartphone app, or the like. The device includes an image capture device, and a programmable memory to execute program instructions, preferably with a network communications interface to access remotely stored information. The device captures an image of a medication dose, whether it be pills, an insulin pen, or a syringe. The device uses image analysis to determine the type of medication and dose, and records the type of medication and dose of medication delivery to the patient, together with a time and date stamp.

The app and device connect to a network to monitor and record the medication delivery times and dosage for a predetermined period of time. The record information can be accessed by or supplied to a caregiver, doctor or other medical personnel, supplier of the medication or manufacturer of the medication and/or delivery device. The app is able to monitor the delivery of the medication to the patient and provide the patient or supplier with information relating to the proper usage. The usage data can be used to calculate a time and amount for refilling of a prescription of the medication and/or delivery device when the patient's supply of the medication is below a predetermined level.

In one embodiment, the delivery device is a pen needle for delivering a dosage of a medication, such as insulin to the patient. The app in the device captures an image of the delivery device and the set dosage of the delivery device. The image is analyzed to calculate the dosage and stores the dosage in the memory. The device is able to capture a sequence of images for each dosage and record the dosages. The dosage data is transmitted to a data storage medium for access by medical personnel, supplier, or the patient. The data is used to calculate a rate of use of the medication and calculate time for resupplying the patient with the medication and to provide a record of the consumed medication over a predetermined period of time.

The app and device provide a method for monitoring and storing data relating the usage and dosage of a medication. The app and device capture and image of the delivery device or medication and analyze the image to determine the specific medication, manufacturer, supplier, dosage, time and date. The data is stored in the device and/or transferred to a storage data medium, such as the cloud. The data is then accessed by medical personnel to monitor the prescribed medication delivery protocol and usage. A supplier can access the data and resupply the medication at an appropriate time based on the usage of the medication over a predetermined period of time. The device can calculate a total usage or consumption of the medication over a predetermined period of time and compare the total usage with a prescribed amount for the predetermined period of time.

In one embodiment, the device is a portable device for determining dose information, comprising an image capture device, a memory to store an image captured by the image capture device, and a programmable memory and processor adapted to execute program instructions, and adapted to analyze the stored image and determine the type and amount of a medication captured in the image.

The features are also attained by a method of recording a medication delivery protocol comprising the steps of capturing an image of a medication or medication delivery device using an image capturing device of a portable electronic device; analyzing the captured image and calculating medication, dosage, and delivery information; and storing the medication, dosage and delivery information. The stored information of the medication, dosage and delivery device are compared with stored information of the prescribed medication and determine whether the dosage complies with the prescribed medication.

It will be understood that each of the preferred or optional features of the various embodiments may be combined with other features and features described in combination with one or more particular features may also be combined with one or more other features of the other embodiments.

These and other features of the invention will become apparent from the following detailed description of the invention, which in conjunction with the drawings disclose various embodiments of the invention.

### BRIEF DESCRIPTION OF THE DRAWINGS

The following is a brief description of the drawings in which;

FIG. 1 is a flow chart showing the device connected to and conveying data to the patient, payer, caregiver, medical personnel, and medical supply facility;

FIG. 2 is flow chart showing the device and app capturing an image of the delivery device and medication

FIG. 3 is a chart showing the data storage medium and access to the stored data;

FIG. 4 is a flow chart showing the app providing data and information;

FIG. 5 illustrates the medical delivery devices as pen needle delivery devices and indicia for identifying the medication, supplier and dosage;

FIG. 6 illustrates the medical delivery device being a syringe in on one embodiment;

FIG. 7 illustrates the medication as tablets or pills and identifying indicia; and

FIG. 8 is a flow chart of the steps of the app and device.

Throughout the drawings, the reference numbers should be understood to refer the like parts, components, and structures.

### DETAILED DESCRIPTION

The invention relates to a device, app, and method for capturing an image, analyzing the image, and calculating information relating to the type of medication, dose of the medication, and time delivered to a patient. The device and app store the data in the device and/or a data storage medium corresponding to the dose delivered to the patient providing a record and history of medication type and time of delivery. The app monitors the future supply needs and times to the patient for refilling of a medication. The information is provided to the patient to assist the patient in monitoring medication dosage and delivery times. The app calculates a total usage of a medication over a predetermined period of time and compares the total usage with a prescribed usage for the predetermined period of time, and determines whether the actual usage is above or below the prescribed amount, thereby providing an indication of proper compliance with the prescribed medication.

The device is a hand held, portable electronic device such and a smartphone including an application (“app”) for processing information relating to the dose of a medication delivered to a patient over a predetermined period of time. The electronic device includes an image-capturing device and a programmable memory to execute program instructions. The device has a network communications interface to access remotely stored information and to direct information corresponding to the analyzed image of the medication and/or medication delivery device.

The app is configured for capturing and analyzing an image of a medication or a delivery device for the medication. The image of the device or medication is analyzed to identify the medication and identify the dose at the time of delivery to the patient. The specific medication and dose are recorded to provide a record of the dose and time of delivery of the medication to the patient. The data is stored in the device and/or delivered to a suitable external storage medium by the network communications interface. In one embodiment, the data is stored in the cloud. The data provides information for calculation of compliance with a medication delivery protocol and usage, and for supplying the patient with a replenished supply of the medication and/or delivery device for compliance with the medication protocol.

The app in one embodiment is able to coordinate the patient's current supply in their possession of mediation and coordinates the dosage take by the patient with the patient's doctor, pharmacy, caregiver, and payer or insurance provider. The app is able to monitor and record the dosage taken by the patient and compare the dosage taken with a proscribed dosage and determine compliance with the prescribed dosage. The app is able to calculate the patient's current supply of a medication, and coordinate with a supplier and arrange for delivery of the medication to the patient at a suitable time to ensure the patient has an adequate supply of fresh medication and/or medication delivery device.

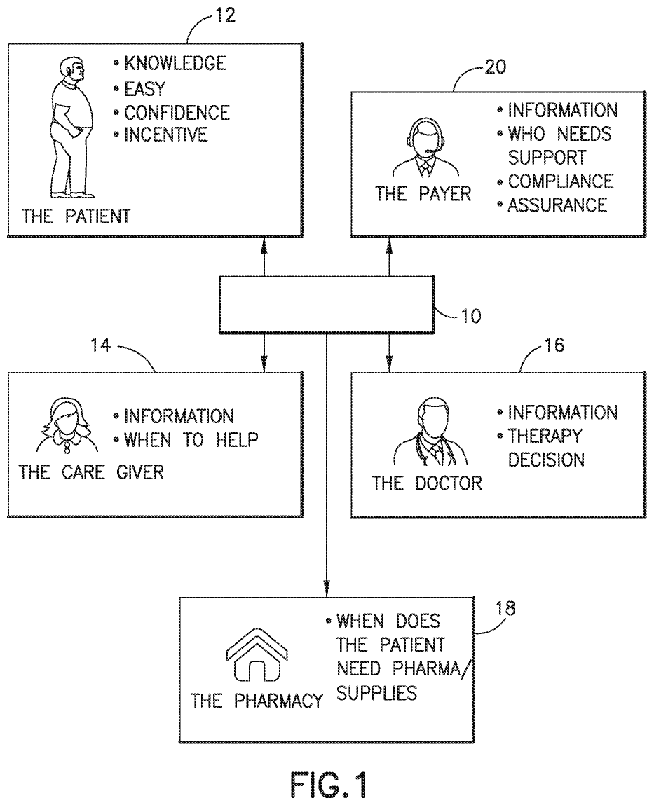

In one embodiment illustrated in FIG. 1, the device 10 is able to capture, store, and transmit data and information relating to the identity of a medication and dosage taken by the patient to an accessible storage medium. Various medical personnel or businesses can access the stored information. The electronic device 10 captures the image and analyzes the image to determine a dosage being delivered to the patient and transmits the data to the patient indicated by block 12 in FIG. 1. In one embodiment the electronic device 10 identifies the medication by the captured image, accesses stored information pertaining to the prescription protocol for the patient and compares the prescription protocol with the current dosage and time for compliance with the prescribed protocol. The information and data is provided to the patient and advises the patient of the specific medication taken and when the medication was taken by a time and date stamp at the time the image is captured. The data is able to notify the patient of the time a subsequent dosage for compliance with the predetermined protocol. The app and device provide information relating to proper compliance with a prescribed delivery time and dosage of the medication. The information and data is conveyed to the patient in a manner providing instant and continuous knowledge of the medications taken and provides the patient with incentives to take the medications in the prescribed manner.

The information and data collected and calculated by the device 10 is conveyed to the caregiver as indicated by block 14 and the doctor indicated by block 16 in FIG. 1 providing a current record of medications, dosages and time of delivery to the patient. The information and data conveyed assists the caregiver and/or doctor in adjusting or modifying dosage amounts, dosage times, and types of medications. The collected information can be conveyed to a pharmacy or supplier indicated by block 18 and the payer or insurance provider indicated by block 20 to determine usage and expected time of refilling a prescribe medication, medical supplies, or delivery device. The payer can then authorize the shipping of the medication and/or delivery device and approve payment to the supplier.

The app is able to provide an incentive or reward to the patient upon calculation of compliance with the prescribed protocol and dosage delivery. The rewards can be, for example, samples of medications or delivery devices or credits for subsequent purchases. The information conveyed to the pharmacy or supplier assists the monitoring of the consumption of a medication and automatically processes a refill of the medication at an appropriate time. The medication can be prepared for pick up by the patient or shipped directly to the patient. The payer or insurance provider is then able to monitor the medication usage and provide payment directly to the supplier.

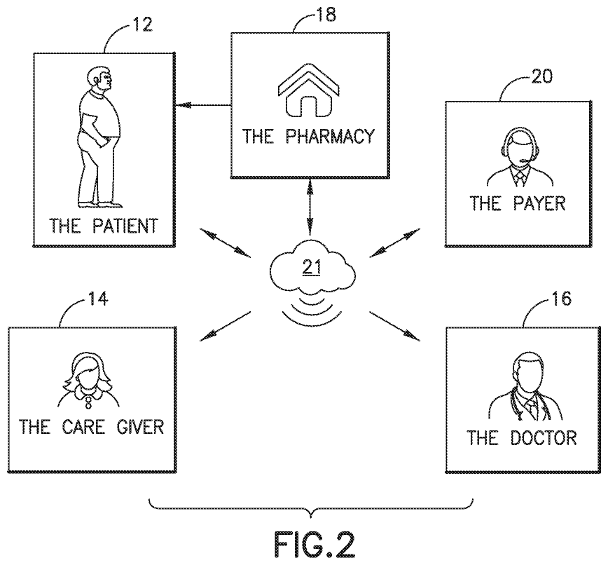

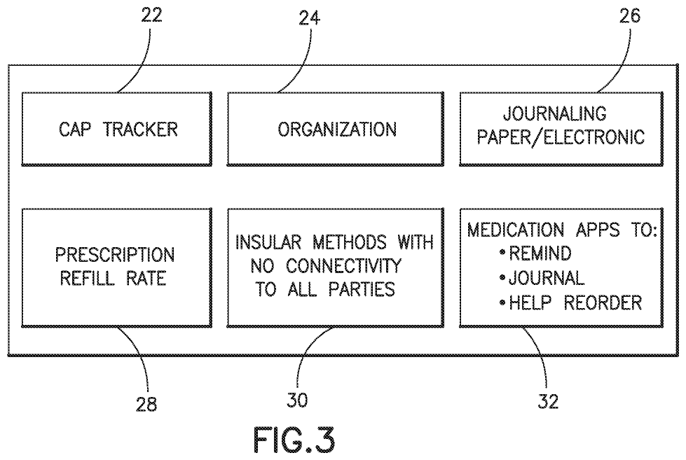

The app as depicted in FIG. 2 and FIG. 3 conveys the analyzed data and information to the external data storage medium, such as the cloud 21 where the information is accessible by authorized individuals. As depicted in FIG. 3, the information is accessible to provide a cap tracker as indicated by block 22 for capturing an image of the cap of a medication container and recording the information indicia on the cap, and an organization indicated by block 24. The information is accessible to provide journaling and recordation of the medication indicated by block 26, prescription fill rates indicated by block 28, medication delivery methods indicated by block 30, and other applications indicated by block 32 to remind the patient of times and dosage, records of past dosage amounts, a usage of the medication to assist in reordering medications and supplies.

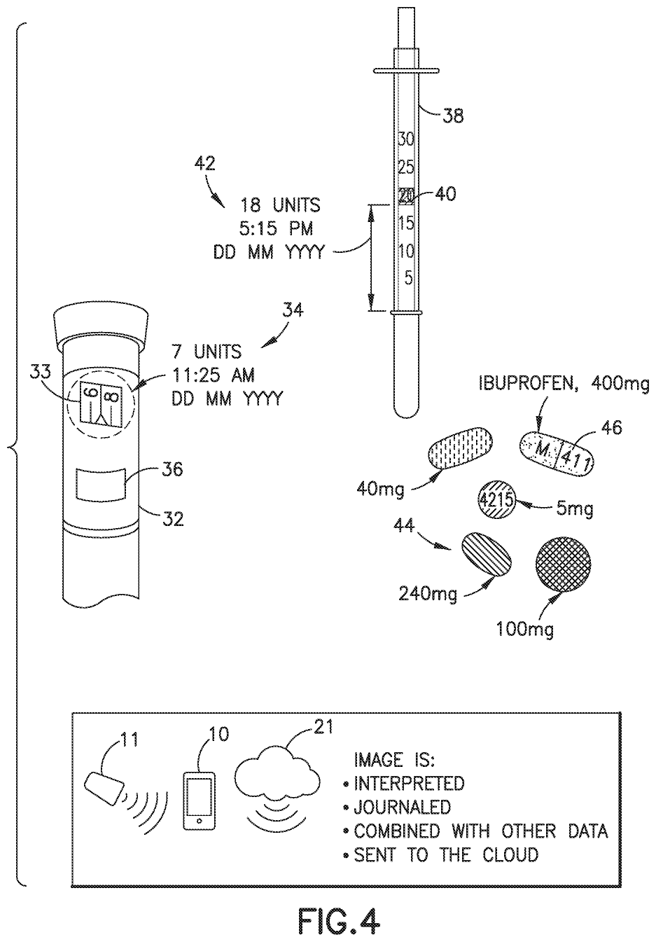

The function and operation of the device and app are depicted in FIG. 4. The device 10 is a portable handheld electronic device, such as a smartphone having an imaging device 11 depicted in FIG. 4 that is able to capture an image of the delivery device and/or medications. It is understood that the imaging is typically incorporated within the device 10. The device 10 captures the image of the medication and/or delivery device and analyzes and processes the image and calculates a dosage being delivered to the patient. The time, date, and dosage are stored in the device 10 and/or transmitted and stored in the external data storage medium.

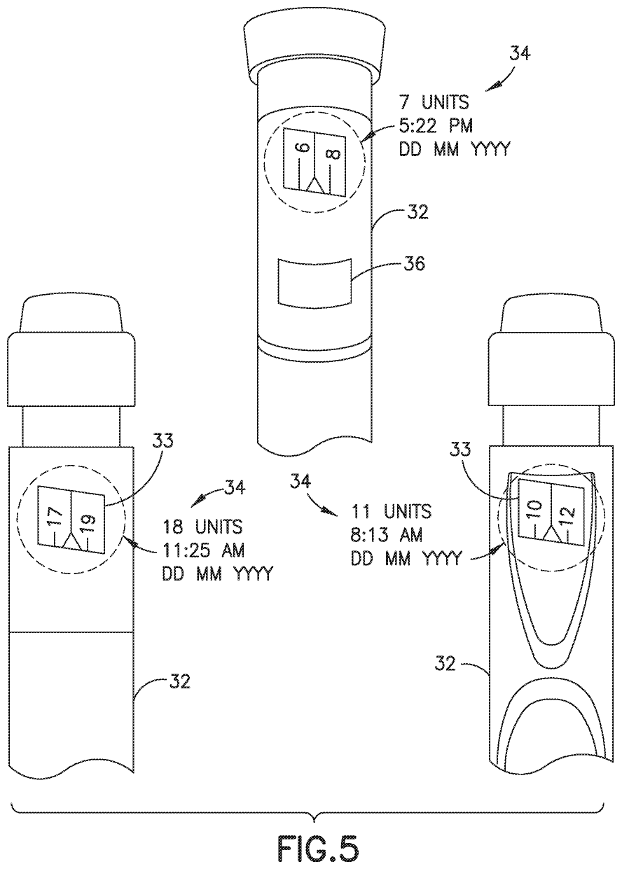

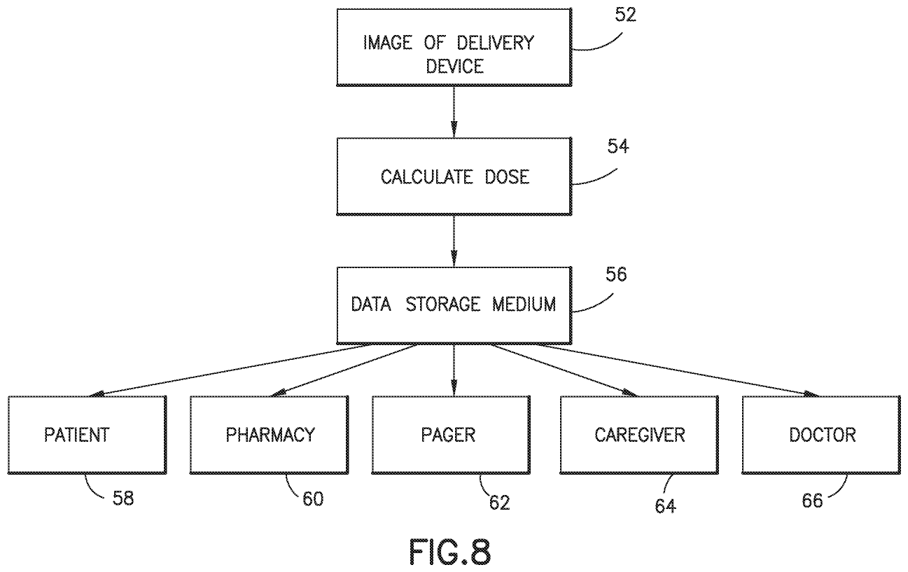

According to an embodiment of the invention shown in FIG. 4 and FIG. 5, device 10 is a smart phone used to capture an image of the delivery device indicated by block 52 and calculate dose information indicated by block 54 shown in the flow chart of FIG. 8. The calculated dose is transmitted and stored in the data storage medium indicated by block 56 where the information is sent to the patient 58, pharmacy 60, payer, 62 and/or caregiver 64 shown in the flow chart of FIG. 8. In the embodiment shown, the delivery device is an insulin delivery pen indicated by reference number 32. The insulin delivery pen 32 in the embodiment shown is a standard delivery pen having a storage compartment and dose knob to select the dosage. The delivery pen 32 includes a dial 33 or other indicia to identify and set the dosage for delivery to the patient. As shown in FIGS. 4 and 5, the dosage is recorded in units as known in the art. A patient first opens the app and captures or snaps a picture of the dialed in dose on the insulin delivery pen 32. The captured image is read, time and date stamped depicted by reference number 34 and stored in the device 10. The data is preferably sent to the external storage medium, such as the cloud where the information can be accessed by the medical provider. The delivery pen 32 in one embodiment, contains an insulin solution. Examples of insulin are sold under the tradename Humalog and Lovolog.

The device captures an image of the delivery pen 32 or other delivery device for each dosage delivered to the patient and records the medication and dosage over a predetermined period of time, such as for example one month. The total consumption or usage of the medication is recorded for the predetermined period of time to provide a complete and accurate record of the medication and delivery. The device 10 can access stored information relating to the prescribed medication amount for the predetermined period of time and compare the total consumption with the prescribed amount. The device 10 can send a notice to the patient and/or supplier when the total consumed amount is at least equal to the prescribed amount for assisting in resupplying the prescribed medication. The device 10 can send a notice to the patient and/or medical provider when the total amount of the consumed medication is below the prescribed amount to advise the patient and medical provider of improper administration of the medication.

The app uses machine vision to identify the dose that is dialed in on the pen 32, depicted in FIG. 5. Advantageously, the manufacture and type of insulin is identified either by a label, bar code, QR code, or other machine readable indicia, shape, color, or other identifying indicia 36 on the delivery pen shown in FIG. 5. In this manner, the proper medication can be verified by comparing the captured image and calculated data with a predetermined medication delivery protocol or device. The information relating to the medication, delivery device, or prescribed protocol can be stored in the device 10 or retrieved from an external data storage medium. If a patient mistakenly picks up a long acting insulin pen, such as that sold under the trade name Lantus, when a short acting insulin or medication dose is prescribed or expected or when long-acting insulin has already been delivered, the app will preferably recognize the unexpected action and provide a notice or warning to the patient providing the patient an opportunity to take corrective measures to administer the correct medication and correct dosage.

By recording and analyzing dose amounts, the app preferably tracks the amount of insulin used for that delivery pen, preforms a calculation to predict when the pen is empty or nearly empty, and alerts the user of the status of the delivery pen or other delivery device. The patient is then able to obtain a fresh supply of the insulin or medication and confirm compliance of the delivery protocol with the medical personnel.

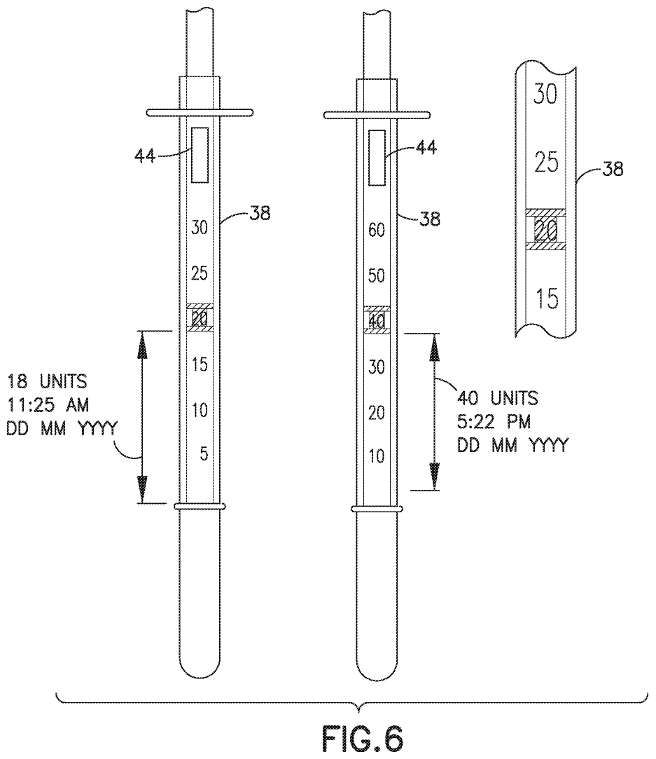

In the case of a syringe 38 shown in FIG. 4 and FIG. 6, the patient opens the app and captures or snaps a picture of the ready to use syringe 36. The syringe can be pre-filled syringe as known in the art. Machine vision analyzes the image of the syringe 38 to determine the plunger location 40 of the syringe relative to the dispensing end of the syringe and calculates the dose, time stamp indicated by 42, and preferably sends the data to the external storage medium, such as the cloud. The app preferably uses machine vision to identify the manufacturer or supplier of the syringe 38 by identifying indicia, size, color and the like. The app is able to read indicia 44 on the syringe to identify the manufacturer and product contained in the syringe 32 as shown in FIG. 6. The app recognizes and calculates a distance of the plunger and stopper from the bottom of the syringe and thereby calculates the dose to be delivered. The app preferably reports the dose to the patient helping the patient to fill the syringe properly to deliver the proper dosage. Where visual markings or indicia are unavailable to correlate with the type of insulin, the app preferably infers what insulin was used knowing what has been prescribed to that patient. The app preferably tracks the amount of insulin used for a given vial and calculates when the patient will need to reorder the insulin, and alerts the patient of the status of the vial or other insulin container.

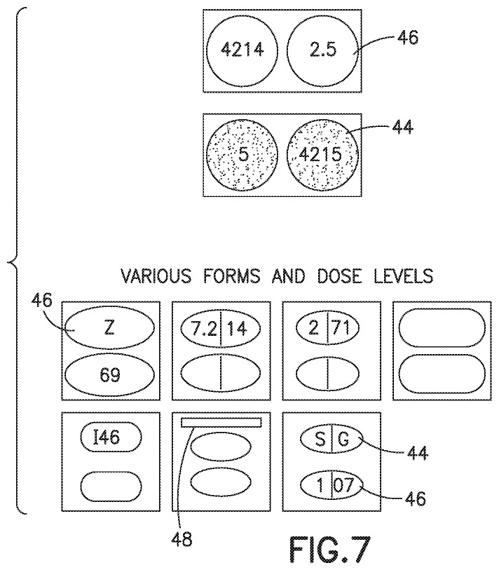

The device and app are also suitable for analyzing an image of tablets or pills 44 shown in FIG. 4 and FIG. 7 and identify the medication and dosages and records the medications and dosages. In the embodiment shown, various medications are illustrated that have different sizes and shapes. The medications and/or packaging can include indicia 46 that can be used to identify the medication and dosage. Examples of medications can be ibuprofen, Atorvastatin, anti-diabetic medications, such as that sold under the tradename Onglyza, and antihypertensive mediations sold under the tradename Verapamil and Losartan.

The patient opens the app and captures an image or a picture of the pills or a group of pills just before taken. The app preferably tracks all prescribes medications the patient is taking and not just those medications for diabetes. The app advantageously visually identifies the pills by manufacturer, supplier, medication, and dosage. The app retrieves medication and dose identifying information based on identifying indicia, shape and color of the pills. The app preferably identifies if the expected pills and the number are present for the prescribed time of day (such as morning vs mid-day vs night). Preferably prior to ingestion of the imaged pills, the app provides an indication to the patient if all is good, or a warning if something is amiss.

The dose is time stamped and the information of the medication or delivery device is preferably stored in an external storage medium, such as the cloud. The stored information can accessed to verify proper dosage and protocol.

The app preferably recognizes whether two drugs are incompatible if, for example, a general practitioner and a specialist have prescribed drugs not knowing what other drugs the patient is already using. The picture provides proof of what was done and a time stamp. Machine vision preferably reads the dose and determines if the dose is correct. The device 10 and the app communicate with a data storage medium to retrieve relevant information to the medication and provide an analysis of the medication for compatibility with other medications and recommended dosages.

A business method may be derived from the above described embodiments. A company may provide the device and/or software to manage patients in exchange for a fee from insurance companies, and deliver improved compliance thus lowering costs to the insurance company due to fewer hospitalizations and complications.

Embodiments of the present invention provide a number of advantages. The system captures with a time stamp insulin doses delivered to the patient, and enables the patient to check when medications were taken and dosage. Embodiments of the invention enable verification of compliance with insulin therapy in addition to other prescribed medications. Embodiments of the invention help patients manage their other complications, identify correct dosage, and identify counter indicated medications. Embodiments of the invention provide proof positive that correct medications were administered, and identify dosing errors before they happen.

In another business method embodiment, patients are provided to a particular pharmacy, in exchange for a fee, for auto-refill when the app determines medication is low.

Embodiments of the invention advantageously increase compliance in any therapeutic area where non-compliance leads to increased costs to payers in either complications or hospitalizations, such as, but not limited to, pulmonary hypertension, MS, and other diseases.

Another embodiment of the invention provides a record of medications given to a patient. This could avoid costly law suits or at least provide crucial evidence beyond just a chart. Embodiments of the invention are preferably linked to an electronic medical record (EMR) providing extra assurance that the right medications are being dispensed. Embodiments of the invention preferably provide a warning if the medications are different in dose or type than indicated in the medical record prior to the medication being dispensed to the patient. Since machine vision is flexible, bar codes or other symbols could be used to further enhance the use of the app.

The above description of the preferred embodiments is not to be deemed as limiting the invention, which is defined by the appended claims. The disclosure is intended to enable the artisan of ordinary skill to practice variants of the invention described without departing from the scope of the invention. Numerical limitations herein, in the specification and in the claims, are understood to be limited by the modifier “about,” such that minor departures yielding equivalent results is within the scope of the invention. Features or dependent claim limitations disclosed in connection with one embodiment or independent claim may be combined in another embodiment or with a different independent claim without departing from the scope of the invention.

1. A portable device for determining dose information, comprising:
an image capture device for imaging a medication or medication delivery device;
a memory to store an image captured by the image capture device; and
a programmable memory and processor adapted to execute program instructions, and adapted to analyze the stored image and determine the type and amount of a medication captured in the image.

2. The portable device of claim 1, wherein the medication delivery device is an insulin delivery pen, and the image is analyzed to determine the type of insulin delivery pen, and a set dose is determined from the insulin delivery pen dose window.

3. The portable device of claim 1, wherein the medication delivery device is in a syringe, and the image is analyzed to determine the dose amount from a syringe plunger location in the syringe.

4. The portable device of claim 1, wherein the medication is one or more pills, and the image is analyzed to compare the pill images with a database of pill shapes and colors to identify the pills and medication concentrations.

5. The portable device of claim 1, wherein said device is configured for capturing a sequence of images corresponding to a sequence of dosages over a predetermined period of time, analyzes the captured images and calculates a medication dosage, and stores each medication dosage.

6. The portable device of claim 1, wherein said device is configured for calculating a total usage of the medication for the predetermined period of time, and compares the total usage of the medication with a prescribed usage for the patient, and sending a notice to the patient when the total usage is at least equal to the proscribed usage or less than the prescribed usage.

7. A method of recording a medication delivery protocol comprising the steps of:
capturing an image of a medication or medication delivery device using an image capturing device of a portable electronic device; analyzing the captured image and calculating medication, dosage, and delivery information; and storing the medication, dosage and delivery information in a data storage medium.

8. The method of claim 6, further comprising capturing a sequence of images corresponding to sequential dosages to a patient over a predetermined period of time, analyzing the captured images and calculating each medication dosage; and storing the dosage information.

9. The method of claim 8, further comprising calculating a total usage of the medication over the predetermined period of time and comparing the total usage of the medication with a prescribed usage corresponding to a supply of the patient, and sending a notice to a medication supplier when the total usage is at least equal to the prescribed usage for providing a new supply of the medication to the patient.

10. The method of claim 9, further comprising sending a notice to the patient when the total usage is below the prescribed usage.

11. The method of claim 9, further comprising sending a notice to the medical personnel when the total usage is below the prescribed usage.

12. The method of claim 9, wherein the medication or medication delivery device includes indicia corresponding to at least one selected from the group consisting of a manufacturer, type of medication, and dosage, and capturing an image of the indicia and analyzing the captured image, and recording the manufacturer, type of medication, or dosage.

13. The method of claim 9, wherein the medication delivery device is a pen needle delivery device, and said method further comprises capturing an image of a dosage indicated on the pen needle delivery device and recording the dosage.

14. The method of claim 9, wherein the delivery device is a syringe having an plunger, and the method comprises capturing an image of the syringe and plunger, analyzing the image and calculating a position of the plunger corresponding to a dosage delivered to the patient, and recording dosage.

15. The method of claim 9, wherein said medication is a tablet or pill, and said method comprises capturing an image of the tablet or pill, and analyzing the image to determine the medication and dosage, and recording the medication and dosage.
